(19) United States
(12) Patent Application Publication
(10) Pub. No.: US 2002/0143241 A1
(43) Pub. Date: Oct. 3, 2002

(54) Method and device for wireless transmission and processing of psychophysiological data

(76) Inventor: Lars-Hakan Thorell, (US)

Correspondence Address: SWIDLER BERLIN SHEREFF FRIEDMAN, LLP, 3000 K STREET, NW, BOX IP, WASHINGTON, DC, 20007, US

(21) Appl. No.: 09/891,604

(22) Filed: Jun. 27, 2001

Related U.S. Application Data
(60) Provisional application No. 60/214,635, filed on Jun. 28, 2000.

(51) Int. Cl. G09G 5/00; A61B 5/00; G09B 19/00; G06F 19/00; G06F 17/00; A63F 9/24; A63F 13/00

(52) U.S. Cl. 600/300; 345/751; 434/236; 463/42

(57) ABSTRACT

The devices and methods of the present invention includes a communication network system that may be used to measure, analyze and distribute psychophysiological and other subject related data in real time from living subjects, such as human beings, or other data sources, who may be close to each other or scattered at different locations. The communication of psychophysiological information or devices may be bi- or multi-directional for psychophysiological intersubject interaction, so that two or more subjects may interchange their psychophysiological data after processing in one or more steps by the means of platforms of different kinds. The representation of processed physiological or other data for oneself or other subjects may be effectuated in different symbolic, functional or physical ways.

### TECHNICAL FIELD

This invention relates to a method and device for measuring, wireless transmission in real time over Internet or other net and processing of psychophysiological data, such as psychic events and states that may be traced in physiological systems.

### BRIEF DESCRIPTION OF THE INVENTION

The devices and methods of the present invention includes a communication network system that may be used to measure, analyze and distribute psychophysiological and other subject related data in real time from living subjects, such as human beings, or other data sources, who may be close to each other or scattered at different locations. The communication of psychophysiological information or devices may be bi- or multi-directional for psychophysiological intersubject interaction, so that two or more subjects may interchange their psychophysiological data after processing in one or more steps by the means of platforms of different kinds. The representation of processed physiological or other data for oneself or other subjects may be effectuated in different symbolic, functional or physical ways.

Psychophysiological intersubject interaction may include, but is not restricted to, situations like

(a) Digital games, such as action games, strategic, role, sport, racing, and family games, childrens' games or any other type of digital game to which psychophysiological signals may be implemented as active parts or added to in stand-alone embodiments. For example: the heart rate and the sweat gland activity may be implemented in interactive digital games over net as conditions, i.e. some tasks cannot be performed unless a subject meets certain psychophysiological conditions, or some processes of the game or some characteristics of the role figures or objects, or some characteristics of the environment in the game, will be modified or exchanged depending on the players own or other players' psychophysiological conditions, or, own and others' psychophysiological signals will be displayed in suitable ways to oneself or to others.

(b) chatting, i.e. written or vocal or symbolical exchange of information over a net between two or more subjects for interpersonal communication. For example: When two or more subjects are communicating the concomitant psychophysiological reactions may be displayed in some suitable way so the subjects may draw some conclusions about the others. The ability to cheat is not a limiting factor for the use of the method since the uncertainty of the communication in chatting is one of the charms and a normal prerequisite of the phenomenon of chatting.

(c) group conversation, such as, for example, interactive role playing, theatre, drama or any other similar situation

(d) other cultural situation such as interactive happenings, arts exhibitions or similar

(e) entertainments of type interactive television programs including talk shows, musical entertainments, contests, interviews, political events

(f) emotional strategy games or exercises—new forms of entertainment that is made possible by the present invention.

(g) any other situation in which two or more subjects want to reveal their psychophysiological reactions or states for each others.

Other subject related data may include other media like

(a) sound in real time

(b) pictures, film, video, web camera pictures in real time

(c) smell

(d) any other medium by which a sensation can be transmitted over net

Other data sources may be

(a) log data, i.e. data encoded, for example by the usage of unit 14 in the system 10, for descriptions of observed or felt reactions, states, behaviour or situations in which the subject is.

(b) time data

(c) positioning system devices

(d) light exposure, such as direct, reflected, white or other qualities of light

(e) ambient temperature

(f) winds, air humidity or other climatic circumstances

(g) sound exposure, such as sound levels

(h) mechanical forces of different kinds

(i) movement characteristics, i.e. acceleration and similar any other environmental data that are relevant for the situation

Platforms used may be such as

(a) computers, i.e. stationary, portable, handheld or other types

(b) game consoles, i.e. special hardware design mainly for digital games.

(c) mobile phones, or other communication devices using local or long distance or satellite telephone nets.

(d) mobile intelligent screens or electronic books or other similar communication devices for local or net communication

Networks may be

(a) Internet or other world wide web and related net functions as networks connecting to Internat from mobile telephones.

(b) Intranets of different types

(c) Local nets, such as within a home or neighboring houses or vehicles or similar

(d) micronets, such as connecting a computer or mobile phone with other devices, or, connecting one or more small devices like the measurement unit 12 and log unit 14 and other.

Symbolical representation of processed physiological or other data may be effectuated by changes on a computer screen or other visual medium in

(a) pictures

(b) verbal expressions in text or sounds

(c) diagrams

(d) colors

(e) acoustich codes/signals

(f) any other symbolic way

Functional representation of processed physiological or other data may be effectuated by changes in the function of

(a) computer program or routine, such as the processes of a computer game or other

(b) mobile phone or other communication device

(c) any other device or process that brings information to a subject.

Physical representation of processed psychophysiological or other data may be effectuated by changes in physical effects by devices applied to the subject's body by

(a) vibrating devices (as, for example, in some mobile telephones)

(b) tapping devices (simulating for example the heartbeat

(c) mechanical effector devices such as devices exerting mechanical pressure or movement to the subjects

(d) heating or cooling devices

(e) electrical devices

(f) any device giving some type of sensation to the subject.

Additional aspects (applications) of the invention. Of course, the applications of the devices and methods of the present invention are not restricted to psychophysiologically intersubject interactive situations. For example, in embodiments including a mobile phone or palm held computer or similar, with the possibility of connections networks, one or more subjects' psychophysiological or other data together or not with data from other sources, may be monitored at close or far distances in mobile or non-mobile isolated or non-isolated situations in the contexts of

(a) Psychological research/clinic, such as tele-psychotherapy, tele-dream analysis, Biofeedback, Autodesensibilization

(b) Sociopsychological research/clinic

(c) Psycho/behavioural therapy or other psychological psychiatric therapy. For example, when a patient is moving freely in the society or over wide landscapes or sea, and the measurement unit detects signs of anxiety the communication over a mobile telephone of the physiological signals in combination or not with verbal communication to a therapist or computerized device may enable suitable counteractions against the anxiety.

(d) Depression- or suicide research/clinic

(e) Menopause research/clinic

(f) Traffic research

In addition, the devices and methods of the present investigation may be used when the psychophysiological activity/reactivity of a multitude of subjects, gathered or scattered, should be monitored simultaneously, which has not been possible before. Such situations may be

(a) commercials research/evaluation. For example: Subjects scattered over many countries receiving commercials over a television satellite may send their psychophysiological reactions over a net to receiving aggregating computers which in turn may send the aggregated data to central computers for final analyses of culturally and in other ways varying subjects' reactions.

(b) music contests evaluation/monitoring. For example: many subjects' psychophysiological reactions may be measured and distributed over net for analyses evaluating the psychophysiological impact of different appearances. The subjects may be seated in a concert hall or by the television sets.

(c) film, video or television or similar evaluation/monitoring

(d) film, theatre, arts, happening contests/evaluations. For example: the actor' psychophysiological status may change the scenery and the audience's reactions may presented in different ways to the actors or change some qualities in the play.

(e) monitoring a group of gathered or scattered athletes with or without the possibility to coaching, for example, the monitoring of a group of cyclists in a long cycle race with a global positioning system offers the possibility to compare the cyclists' psychophysiological status and positions on the road, and to give orders who is going to which relative place in the group. Another example is mass athletic events like the “Vasa race” or similar mass or mrathon races when the interest is to monitor a multitude of individuals' psychophysiological status. The psychophysiological data may be accompanied by environmental data or positional data.

Further, the devices and methods of the present invention may be used for pedagogical or self-knowledge purposes, for example for

(a) recommended psychological experiments using standard monitoring programmes

(b)planned psychological experiments with special programmes

(c) activity monitoring for choice of best time for learning or other activities

(d) the study of own reactions to certain events, stimuli or objects. Can be used for the study of what evokes psychophysiological reactions

(e) monitoring of own or others' psychophysiological signal during the day or 24 h periods.

(f) monitoring sleep

(g) own dream studies

(h) ergonomic studies. For example: the electromyogram from a certain group of muscles may be monitored, and when the computer detects an increased muscle tonus according to criteria, an alarm signal or a computer process may be initiated for a defined counteraction from the subject's side.

(i) monitoring any activity, for example jogging, mediation, car driving, sun bathing etc. etc. For example: a global positioning system together with environmental data (temperature, sound level or other) and psychophysiological information may be recorded simultaneously from one to several car drivers in interactive driving situations in the traffic during long time and over long distances and sent over mobile telephones and WAP and Internet or telephone net, enabling simultaneous analyses in central receiving computers.

(j) monitoring and controlling stress/anxiety or embarrassment situations in group or net situations.

(k) Call-up-alert functions such as when certain psychophysiological conditions are met, an alert signal may go to a computer or /mobile/ phone to call up a certain telephone number to a receiving computerized device or a person.

(l) Monitoring isolated persons' psychophysiological status.

### BRIEF DESCRIPTION OF THE DRAWINGS

FIG. 1 is a schematic system diagram of the units of the present invention; and

FIG. 2 is a schematic flow diagram of the main processes and structures within the units of the present invention.

FIG. 3 is a schematic flow diagram of the signals transmitted within the most preferred solution P of the present invention.

FIG. 4 is a schematic flow diagram of the signals transmitted within the secondary solution S of the present invention.

### DESCRIPTION OF EMBODIMENTS

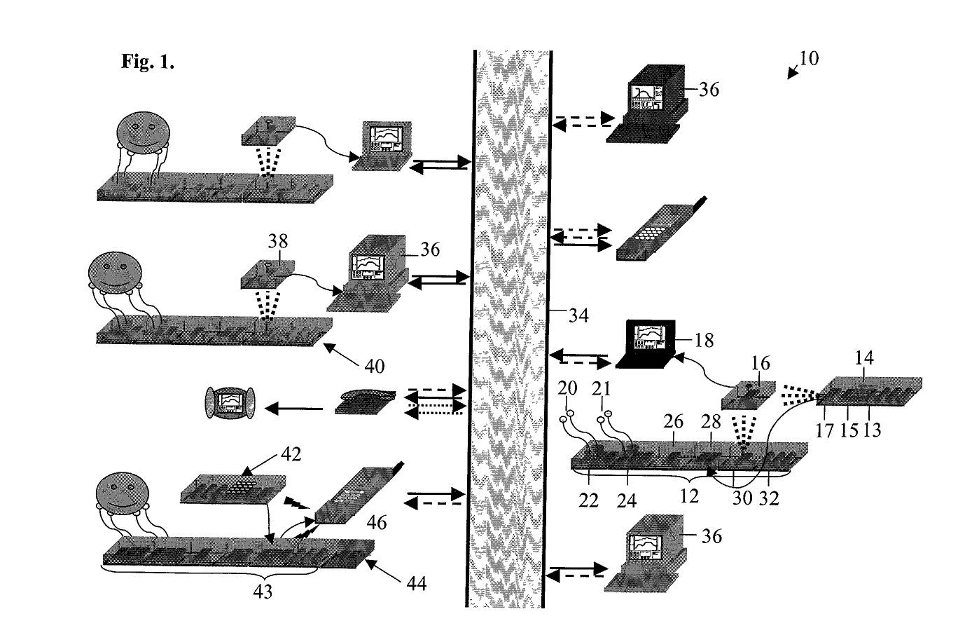

With reference to FIGS. 1-4, the device and method of the present invention includes a communication system 10 that may be used to measure and analyze psychophysiological signals or data from living subjects such as human beings or animals. The communication of psychophysiological information may be sent in one direction to analyzing device or devices or may be bi-directional for psychophysiological interaction, so that two or more subjects may interchange their psychophysiological data. Such a psychophysiologically interactive situation can be deducted from FIG. 1 in which the example comprises an interaction over the units 18, 36, 46 from three subjects to three subjects.

The system 10 is a very small portable measurement- and distribution system for psychophysiological signals via wire or wireless transmission from one or many transmitting subjects to one or many subjects in both directions or one. The subjects may be geographically scattered or gathered and the transmission may occur via mobile telephones or similar devices, or via computers that may be connected to the Internet or other networks of transmitting and receiving devices or to one or many approved receiving subjects' devices including data processing in real time in one or many steps for a great number of global or local applications including, but not limited to, entertainment, scientific, commercial, public, sports, private, clinical, educational and many other applications.

The psychophysiological signals may include, but are not restricted to, electrical brain activity, sweating, heart rate, blood pressure, blood flow, respiration, eye movements, electrical and mechanical muscle activity, acceleration of movements and other such activities of a living subject. Certain behaviors, such as concentration, anger, emotional engagement, stress, physical exertion and relaxation trigger often different psychophysiological signals that may be analyzed.

In other words, the psychophysiological signals may be correlated with conditions of the subject who is measured. For example, eye movements and sweating may be analyzed together to find out how a viewer reacts to a television commercial. The eye movements may inform the advertiser what parts of the advertisement that catches a viewer's attention and the sweating may indicate the level of engagement and attention of the viewer. Another example is an on-line computer game between two or more subjects over a net in which the players' psychophysiological or other data through the system 10 are transmitted to the net and further to the other players, thus allowing the players' emotional reactions in the form of heart rate and sweating or other type of reactions influencing the qualities of the game and role characters and revealing the reactions for all or some subjects. A third example is a situation called “chatting” during which two or many subjects exchange verbal information, written and/or acoustically with or without web camera or other picture generated visual signals over a network, with the addition of their psychophysiological reactions in a similar way as in the example of on-line computer games over a net. Preferably, the system 10 includes a very small and lightweight measuring unit 12 and an optional log unit 14 that are in communication with a transit unit 16 that may be separate or part of a computer 18, of a mobile telephone 46, or of other communication device. As mentioned below, the units 12, 14 may be communicating directly with another. An important feature of the present invention is that the communication system 10 may be in wireless communication with other communication and computer systems, also described in detail below. The system 10 is fully compatible with any currently available computer and communication devices.

The measuring unit 12 and the log unit 14 may be in communication with the transit unit 16 via infrared transmission, short wave radio technology (such as “Bluetooth communication” or other) or any other suitable communication medium. This means that psychophysiological data from several subjects, who may be at different locations, may simultaneously be analyzed and results be redistributed to anyone thus enabling situations of psychophysiological interaction. Of course, the communication may be via wires and other suitable links also.

The measurement unit 12 has a plurality of sensors 20 that may be attached to any part of the body of a subject to be measured or analyzed. Because the unit 12 is small, it may be placed relatively close to the source of the signals which may reduce any interferences. The type of sensors and amplifiers may be adjusted to what is being measured. For example, electrodermal activity (EDA) (sweat gland activity) may require electrodes of certain types and a DC voltage of about 0.5V compared to the measurement of brain activity that may require other types of electrodes and passive filtered amplification of bioelectrical AC signals. Breathing and other conditions may require other filtering amplification characteristics.

The measurement unit 12 has several modules including signal amplifiers 22 that modulate and amplify signals received from the sensors 20. The unit 12 may also have a second set of amplifiers 24 that may be designed to measure other psychophysiological data such as for example blood flow. The amplifiers may be adapted to be cooperating with a set of sensors 21. Of course, the measurement unit 12 may include additional sets of amplifiers for similar or different purposes, if necessary.

The measurement unit 12 may also have a process module 26 including, for example, a start/activation unit 70, a timer unit 72 including analog to digital converter functions, a basic data unit 74, a final logics unit 76 including, for example, buffer handling, a stop unit 78 and an optional reset unit 80. The signal-formatting module 28 may be used to format the digital signals from the measurement modules 22, 24 or other extra measurement modules, and from the process module 26, or from other sources such. The formatted signals from the signal formatting unit 28 may be fed into a buffer at a certain rate, decided by the process module 26, if needed, or continuously, to the transmitter module 30, which may transmit the information out from the set of units 12 and 14.

The transmission information may use infrared transmission, radio transmission (for example of type Bluetooth) or any other suitable transmission system, including wire, forward over a unit 16 or directly to a stationary or portable computer or other computerized device, which regardless of type of device is connected to the Internet or other type of net. For example, the transmission information may also be transmitted to a mobile telephone or a palm held computer or portable computerized screens or another portable device that is connected to the Internet or other type of net. The connection to mobile telephones and other portable devices is of particular interest because the subjects to be measured may be freely moving about anywhere in the world if the mobile phone or other device is connectable to the Internet or other net. The subject wearing the measurement unit 12 may connect the computer or other computerized device or the mobile telephone to a suitable web site or remote computer prior to starting a suitable application program to initiate the web-site and prepare the web-site or any other receiver to receive the psychophysiological information that is transmitted by the set of units 12 and 14.

The measurement unit 12 has also a power supply module 32 for supplying the unit 12 and sensors 20, 21 and others, if needed, with the necessary power. The unit 12 may, optionally, also be equipped with a module 44 containing for example a positioning system, such as global positioning system (GPS) that indicates the geographical position of the subject wearing the measurement unit 12.

Two or more measurement units of type 12 with one or more amplifiers each applied to one or more subjects can connect to a ‘micronet’. The micronet can include a local signal receiver like the transit unit 16 of a computer 18 or of a mobile telephone or of any communication device for further transmission to a net. Such a solution will, for example, enable the measurement and sending electromyographical (muscle activity) signals from left and right legs and arms separately to a mobile telephone or other device, without uncomfortable wire interconnections between the extremities. In addition, such a micronet enables situations of psychophysiological interaction locally without a transmission via the Internet.

The optional transit unit 16 may be adapted to demodulate the signals received from the transmitter module 30 so that the transmitted data is in a format that may easily be used in the computer 18, mobile telephone 46 or other communication device. As mentioned above, preferably, the unit 16 is an integral component of a computer such as 18, 36, mobile telephone 46 or other communication or computer device. Alternatively, depending on the type of data format, the transit unit 16 is not needed and the transmission information is fed directly to an existing input port of the computer, mobile phone or other communication or computer device in connection with a net such as for example the Internet. An important feature of the unit 16 is that it may simultaneously receive information from several sources, for example, a module 30 and a log unit 14.

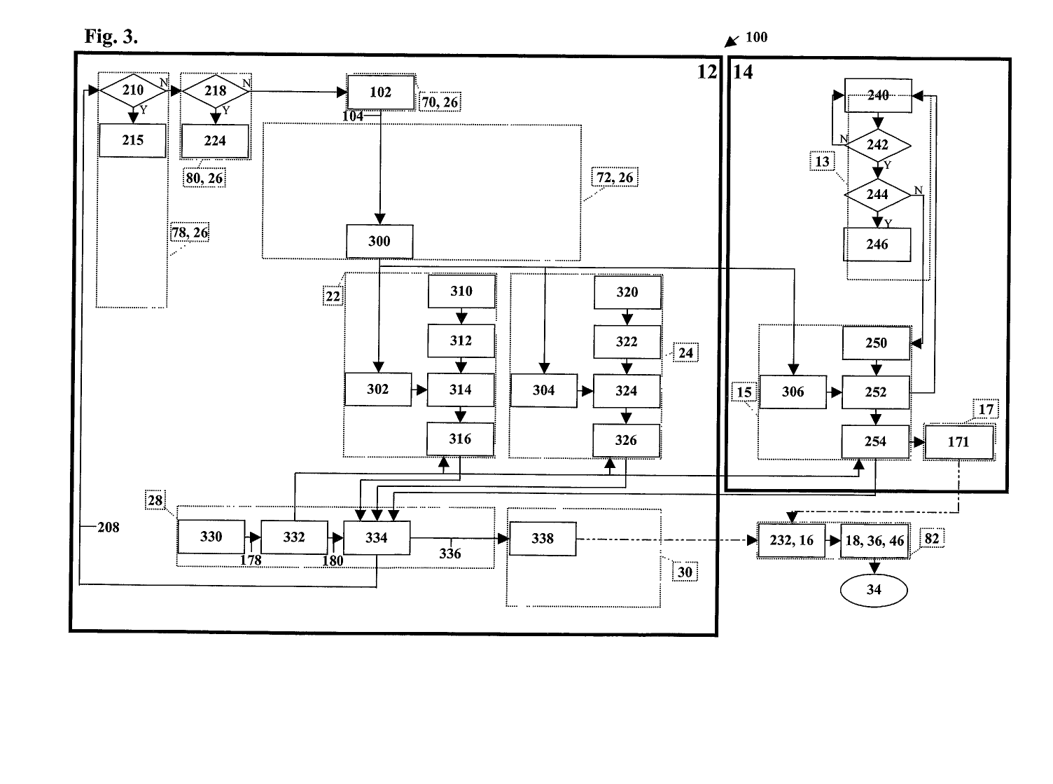

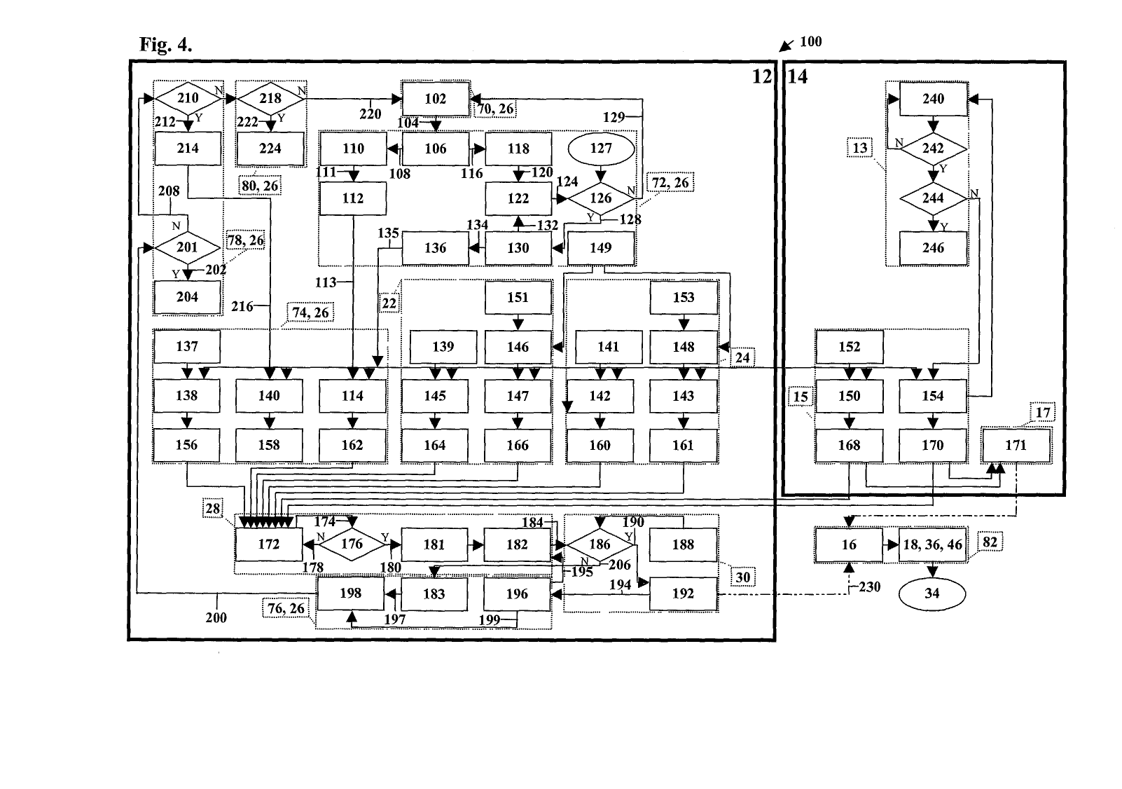

In this way, the log unit 14 may be used to simultaneously transmit information to the transit unit 16 together with the transmitter module 30. Alternatively, the information from the log unit 14 can be sent to the module 28 in the measurement unit 12, a solution that is described in FIG. 3 (communication from unit 254 to unit 234, solution P) and in FIG. 4 (communication from unit 14, i.e. the ID register unit 168 and the data register 170 to the validity register unit 172). One advantage of simultaneously using the log unit 14 is that an observer may observe the subject connected to the sensors 20, 21 or other sensors and enter behavioral or environmental patterns such as anger, smile, provocation, external events or states etc. and transmit the information so that the information from the log unit 14 may appear in the same data body as the information transmitted from the measurement unit 12. The use of a unit 14 or similar makes it easier to interpret the information transmitted by the unit 12. Alternatively, the subject being measured may him/herself enter own or others behavioral or environmental patterns via the log unit 14 and further.

The log unit 14 may have a log key board module 13 consisting of a numerical key pad with coded messages so that a first button may be associated with a certain behavior such as anger, facial expression, provocation, calmness, physical aggression, crying, running away etc. A second button may be associated with a different behavior and so on. The current combination of coded keys may be held in a data module 15 which continuously is available to a transmit module 17 for wired or wireless continuous or timed transmission to a transit unit 16 or to a signal formatting module 28 of the measurement unit 12. The coded message from the log unit 14 may be matched with the information from the measurement unit 12 to confirm the if behavior of the subject to be measured by the unit 12. The log unit 14 may also provide information about the status of the subject prior to the observed behavior as measured by the measurement unit 12. Perhaps the subject was calm prior to the provocation.

When a computer 18 or other communication device receives the information, the device optionally may synchronize the information which may include identifiers so that it is possible to identify which of the measurement units 12, if there are more than one unit 12, that transmitted the information. Similarly, the computer 18 or other communication device optionally may identify which of the log units 14 that transmitted the information so that the transmitted information may be associated with the correct subject to be measured.

The format of the data may enable communication from unit 30 to unit 16 in connection with a computer 18, mobile telephone 46 or other communication device, in a way that is independent of ‘handshaking’ based communication protocol. This construction avoids problems in reestablishing communication after unwanted communication interrupts.

The computer 18 may be connected to the Internet or other net 34 so that the information may be sent to one or more other computers 36 that is disposed remotely, thus enabling situations of psychophysiological/emotional interaction and/or aggregated analyzes in one to several steps including one to several levels of information processing and/or groups of connected subjects with units 12 and/or 14. The computer 36 may be any suitable device including stationary, laptop or handheld or other computer, mobile phone or any other computerized communication device.

An extra module, the measurement module 44, may be used for collecting extra data such as, for example, light exposure, sound level, mechanical pressure and/or GPS data including clock time and positioning information. The module 44 may be treated as one of other measurement modules of type 22, 24. A log unit 42, that is similar to the log unit 14, may transmit information via the measurement unit 43, that is similar to the measurement unit 12, directly to a mobile telephone 46 that in turn may be in communication with the Internet or other net 34. The mobility of units 42, 43, 44, may, for example, be used to analyze the training of athletes so that the psychological information is continuously transmitted to a coach for further analysis of the condition of the athlete and several athletes in real time. In other words, the flexibility of the system 10 permits the measurement of psychophysiological signals in real time from one or many independently moving subjects or subjects that may be very remote from another.

In an alternative embodiment, the measurement unit 12 may be a stand-alone biofeedback device that is not required to be connected to any computerized external device at all. The measurement unit 12 may, for example, have output possibilities for an acoustic signal that is proportional in frequency to the magnitude of a selected signal channel for the feedback. The measurement unit 12 may also have a digitally based visual messenger, for example, a small digital display, that conveys information related to the absolute or relative value of one or more selectable signal channels for the feedback. The display may, if equipped with suitable devices, such as GPS, show the exact time and the position in digital form and/or on a map. The measurement unit 12, log unit 14, transit unit 16 and computerized device or phone 18 may be used locally without being connected to the Internet and in such stand-alone configuration be useful in a wide range of applications.

In another alternative embodiment, the measurement unit 12 may send data via wire or via wireless communication, such as infrared or radio signals (for example, Bluetooth) or other communication means to external devices like, for example, sound equipment (such as, for example, head phones or sound amplifiers), visual equipment (such as, for example, digital displays, TV sets or projectors) or any equipment exerting sensory activation such as, for example, vibrating devices (as, for example, in some mobile telephones) or tapping devices (simulating the heartbeat or other physiological signals) or shifts in mechanical pressure or temperature applied to the subject's body or nerve stimulation devices through mechanical, electrical or magnetic stimulation of a subject's senses or nervous system.

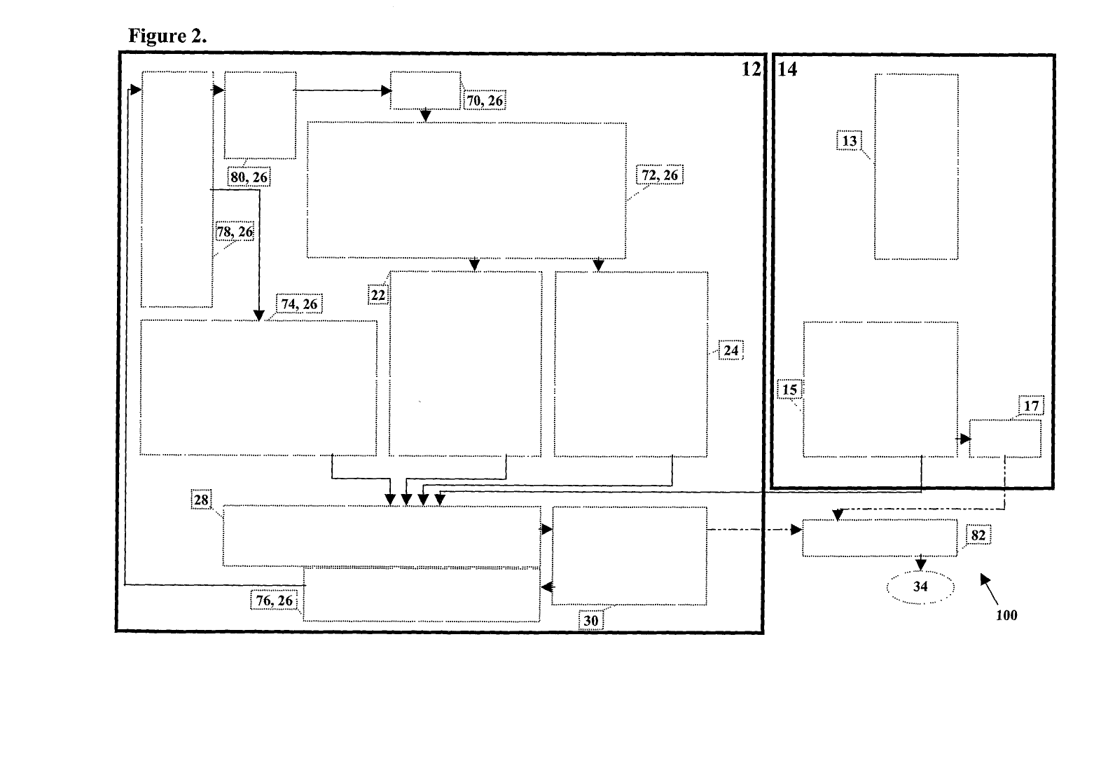

FIG. 2 shows the main processes and structures of a schematic flow diagram 100 of the signals that are transmitted when the measurement unit 12 and the optional log unit 14 are in operation. FIG. 3 displays the most preferred solution (P) of that main processes and structures because of its simplicity, low cost and the possibility to shrink its size extremely much. FIG. 4 contains a secondary solution (S) that offers very high fidelity of the digital signals to the receiving equipment to the original analog signals from the subject. The two solutions P and S are mutually exclusive within one single set of a measurement unit 12 and a log unit 14, but can be mixed within a measurement system 10 consisting of two or more sets of units 12 and 14.

First, the FIG. 2 regarding the main structures and processes is explained, then the most preferred solution P, and last the secondary solution S.

In FIG. 2, showing the main structures and processes in the measurement system 100, a start/activation unit 70 of the measurement unit 12 sends, from when the start button has been pressed, may set a continuous activation signal 104 high to a timer unit 72 of the process module 26. The timer unit 72 sends at regular intervals time pulses that govern the timing of processes in the measurement modules 22 (measurement channel number 1, ch1) and 24 (optional measurement channels number 2, 3, 4 up to an unspecified number of measurement channels). The timer unit 72 also influences processes, directly or indirectly, in the log unit 14, and the signal formatting module 28. Further, processes in the transmitter module 30 may be timed by the timer unit 72.

The measurement unit 22 (ch1) and optional measurement channels 2 etc. (ch2 etc.) in the measurement symbolically represented by the module 24, includes amplification and signal conditioning functions specific to the type of signals measured through the sensors 20, 21. The units 22, 24 etc. provides digitalized information from analog to digital converters of different kinds and varying capacity regarding resolution in magnitude and time.

The optional basic data module 74 contains, if present, basic information specifically related to the measurement unit 12. The basic data, if module 74 is present, are, together with the digital information from the measurement modules 22 and 24 etc. and that from the optional log unit 14, if present, available to the signal-formatting module 28 continuously, or at times governed by the timer unit 72. The signal-formatting module 28 provides continuous or timed availability of information in formatted, i.e. encoded, form to the transmitter module 30. The transmitter unit 30 may, in turn, continuously or in a timed way, transmit the encoded information by infrared or radio signals or by wire to receiver units 82 that may be an optional transit unit 16 for further transmission, after suitable data conversion, to a computer 18, 36 input port or a mobile telephone 46 input or other receiving device. The signals from units 18, 36, 46 may further be transmitted to the Internet or other net 34. The optional buffer handling unit 76 in the process module 26 handles possible stop signals and buffer functions. The stop function 78 and the optional reset function 80 of the process module 26 may take care of possible maneuvers from the subject, having pressed a stop or reset button. The process continues-as long as the stop button has not been pushed-further via the start/activation function 72 of the process module 26.

The most preferred solution P is generally characterized by one-bit analog to digital conversion of analog measurement signals and continuous binarily summation of an amplitude-modulated electrical wave, the amplitude of the wave being dependent on the order number of the channel number generating the signal at any moment, and the modulation activated by logical high signals from different information sources such as, for example, amplifiers (one bit high) and buttons (button pressed). Further, the amplitude-modulated signals are continuously added to each other into one single signal comprising the sum of all binarily amplitude-modulated single channels with currently logical signals set high. In addition, the continuously summated signal is also continuously sent further to a transmitter unit preferably designed for, but not restricted to, audio signal transmission.

FIG. 3, shows a schematic flow diagram of the most preferred solution P in detail, illustrating the signals that are transmitted when the units 12 and 14 are in operation. A start/activation unit 102 of the measurement unit 12 may send, from when the start button has been pressed, a continuous activation signal 104 to an analog timer unit 300 of the process module 26. The analog timer unit 300 may send at regular intervals time pulses to the calibration unit 302 in the measurement module 22, calibration unit 304 of units 24 (symbolizing additional measurement channels) and calibration unit 306 for an optional, wired log unit 24. The calibration units send activation signals to the logical signal unit 314 of the measurement module 22, the logical signal units 324 in optional additional measurement units 24, and the logical signal unit 252 of the optional log unit 14 in order to set then logically high, thus simultaneously producing maximal level output signals from the logical signal units 314, 324, 252.

Still describing the most preferred solution P, FIG. 3, the analog signals from amplifiers 310, 320 or similar devices within the measurement units 22, 24 etc., are continuously digitalized by analog to digital one bit converters 312, 322, built on analog technique. Increasing analog signal levels are converted to a logical one (high) signal, equal or decreasing signal levels are converted to a logical zero (low) signal, the logical signals being stored in the logical signal units 314, 324. The logical one-bit signals in the units 314, 324, are continuously modulating a sinus, or other form of, wave by the wave modulator units 316, 326 of the measurement units 22, 24, into amplitudes of the wave different for different measurement modules. The wave is generated in the wave generator 330 and distributed by the wave distribution unit 332 to each of the measurement units' wave modulator units 316, 326. The modulation of the signal in measurement unit 22, i.e. channel #1 (ch1), results in an amplitude of value 1 (arbitrary scale). The modulation of the signal in the first optional additional measurement unit 24, i.e. channel #2 (ch2), results in an amplitude of value 2. The modulation of the signal in the next optional additional measurement unit 24, i.e. channel #3 (ch3), results in an amplitude of value 4. Thus, the amplitude of the modulation is computed by the formula 2ˆ (Ch#-1) units, where “units” is the amplitude of channel #1 and “Ch#” is the order number from 1 up to the order number of an unidentified maximum number of channels in the measurement unit 12. In a similar way, each of the keys in the keyboard module 13 of the log unit 14 can be seen as separate channels ch2, ch3 etc, or ch3, ch4 etc if there are two measurement modules 22, 24, or, ch4, ch5 etc if there three measurement modules 22, 24 and similar. Thus, each current key code in effect in the key code register 250 results through the logical signal unit 252 and the wave modulator unit 254 an amplitude modulated wave representing the order number of the currently activated key. The units 252 and 254 symbolize similar units up to an undefined maximum of number corresponding to the number of keys available. The wave to be modulated is generated in the wave generator 330 and distributed by the wave distribution unit 332 to each of the log unit's wave modulator units. The amplitude-modulated signal is continuously available in the wave modulator unit 254.

Alternatively, a similar wave generator, distributor and summator as 330, 332, 334, may also be part of the log unit 14 in the case the log unit is not connected by wire to the measurement unit 12 but transmitting by the log transmitter unit 171 in the transmit module 17. The summated signal from the log unit 14 is then transmitted to a receiving system 80 as described below for the measurement unit 12.

Those in the wave modulator units 316, 326, 254 continuously available wave signals are summated by the summating unit 334 in the signal formatting module 28, into one single summated output signal 336 comprising the sum of all binaurally amplitude modulated singles channels with currently logical signals set high. In addition, the continuously summated signal 336 is also continuously sent further to a transmitter unit 338 preferably designed for, but not restricted to, audio signal transmission. The by the transmitter unit 338 sent summated signal 336 may reach a receiving system 82 comprising an optional transit unit 16 and a computer 18, 36 or a mobile phone 46 or other suitable receiving device for analyzes or presentation and may further be transmitted to a net 34.

The summation in the signal-formatting module is followed by a stop button check 210 in the stop function unit 78 in the process module 26. If the stop button has been pressed the process is terminated by the process termination unit 215 which lets the activation signal 104 go low. In the optional reset function unit 80 the reset button check 218 chooses to reset the measurement unit 12 to its original status by the reset process unit 224. If not, the process goes on as long as the activation signal 104 is high.

The measurement unit 14 comprises a log unit keyboard module 13, a log data module 15 and an optional log data transmit module 17. The log unit start/activation unit 240 activates the log key check unit 242 to check whether a log key has been pressed. If not, the log key check unit 242 allows the unit 240 to continue the loop. If the log key check unit 242 determines that a key has been pressed then the log stop key check 244 determines whether the log stop key was the one pressed. If yes, the log stop key check 244 forwards the process to a log process termination unit 246 that terminates the process in the log unit 14. If, on the other hand, the log stop key check 244 finds that the key was not the log stop key, the code of the key is sent to the log key codes register 250 together with current other key codes. The key codes are transformed into logical signals in the way that the first key code is treated as channel #2 in a logical signal units 324, the second as channel #3 etc. Thus, each of the keys in the keyboard module 13 of the log unit 14 is seen as a separate channel. Thus, each current key code in effect in the key code register 250 results through the logical signal unit 252 and the wave modulator unit 254 an amplitude modulated wave representing the order number of the currently activated key. The units 252 and 254 symbolize similar units up to an undefined maximum of number corresponding to the number of keys available. 14 are mutually exclusive. The 254 loaded into the log unit key register 154

The secondary solution S is generally characterized by analog to digital conversion of a resolution by at least 8 bits, preferably higher, of the analog measurement signals. The conversion is typically timed to a high rate. The digitalized signals are kept in registers that, together with basic data such as unit ID:s, time ticks since start of conversion and maybe other information kept in registers, constitute the hardware base for the special data format that is used for each single sample that may or not be temporarily stored in an output data buffer for a timed transmission to a receiver system.

FIG. 4 shows a schematic flow diagram of the secondary, alternative solution S in detail, illustrating the signals that are transmitted when the units 12 and 14 are in operation. More particularly, an activation unit 102 of the measurement unit 12 sends an activation signal 104 to a clock device 106. The device 106 sends an increment signal 108 to an increment device 110. The device 110 sends an increment signal 111 to a time counter 112 and the unit 112 sends a trace signal 113 to a time tracer 114 in the basic data unit 74 of the process module 26 that traces the time from the start of the unit 102. The clock device 106 also sends an interval signal 116 to an interval increment device 118 that transmits an interval increment signal 120 to an interval counter 122 that counts the number of intervals. The counter 122 sends an interval comparison signal 124 to an interval comparison unit 126 that compares the number of intervals to a preset interval value received from a preset interval unit 127. If the number of intervals is equal to the preset interval value then the comparison unit 126 sends an interval equal “yes”-signal 128 to a reset interval counter unit 130 that in turn sends a reset interval counter signal 132 to the interval counter 122. Additionally, the reset interval counter unit 130 sends a “load activate” signal 134 to a central “download all registers” unit 136. If the interval comparison unit 126 determines that the number of intervals is insufficient, then the unit 126 sends a continuation interval equal “no”-signal 129 to the activation unit 102 to instruct the device to continue sending information to the unit 118.

The registers may include a unit 12 ID code 137 that includes an identification of measurement unit 12, loading its content into a unit 12 ID register 138, a unit 12 base register 140 that includes information about which sample interval that is used and data about how the system is configured such as indicating whether the log unit 14 is included or not. The base register 140 also includes information about channel identifiers, number of channels of the data. The system 100 has at least one channel #1 measurement module 22 that may be a unit that is especially designed for electrodermal activity (EDA) that may be received from sweat glands of the subject measured and the channel #1 ID code 139 may be used to identify the hardware unit that is tracing the EDA. The channel #1 ID register 145 holds the channel 1# ID code 159 of the measurement unit 22. The measurement unit 22 is used as an example to illustrate a typical channel, however, other types of measurement devices may also be used. The channel #1 analog device 151 mediates the analog data from the measurement unit 22 amplifier and the channel #1 digital device 146 contains the, by some analog to digital conversion circuit as the analog-to-digital converter 149, digitalized EDA data that is loaded into the channel #1 digital data register 147.

The system 100 may also have extra measurement units up to an undefined maximum number for measurements into channels #2 and up. Such extra measurement units are symbolized by depicting only one single measurement unit 24 in the system 100. The ID of that unit is held in the optional channels ID code 141 which is loaded into the optional channels ID register 142. The optional channels analog device 153 mediates the analog data from the measurement unit 24 amplifiers/amplifiers and the optional channels digital device 148 contains the, by some analog to digital conversion circuit as the analog-to-digital converter 149, digitalized optional data that is loaded into the optional channels digital data register 143. Such data may be, for example, other psychophysiological data, light exposure, integrated sound level or GPS data including clock time and positioning information and other variables. The measurement unit 24 may represent one to several extra optional units measuring several psychophysiological or other variables.

The measurement unit 14 is similar to that used in the most preferred solution P. However, in the present secondary solution S, the hardware log unit ID code 152 is loaded into the log unit ID register 150. The log unit key register 154 includes the digital code that the log unit 14 currently is holding. That code comprises the current key combinations set by key operations at the log unit keyboard module 13.

Preferably, and in the embodiment that the optional log unit 14 transmits its data to the signal formatting unit 28 of the measurement unit 12, the received signals in the registers 138, 140, 114, 145, 147, 142, 143, 150, 154 are downloaded at the “download all registers” signal 135 from the “download all registers” unit 136 to special download registers 156, 158, 162, 164, 166, 160, 161, 168, 170, respectively. The registers may range from at least 8 bits, preferably 32 or higher number of bits, if necessary. All the special download registers each sends a signal to a download validity register 172. The unit 172 is in invalid state until all the registers have sent an acceptable signal. The unit 172 therefore sends a validity signal 174 to a validity comparison unit 176 that determines if all the registers have sent a suitable signal. If the unit 176 determines that not all the registers have sent an acceptable signal, then the unit 176 sends back a validity “no”-signal 178 so that the unit 172 must wait a certain time period until the unit 172 again sends the validity signal 174 to the unit 176. If the unit 176 determines that all the registers have a sent a signal for valid register data, the unit 176 transmits a validity “yes”-signal 180 to a load buffer unit 181 that at the place pointed to by the sample counter/pointer 182 loads the current sample of data held by the special download registers 156, 158, 162, 164, 166, 160, 161, 168, 170, and possibly other registers belonging to other modules, such as, for example, unit 24 for ECG, respectively, into the data buffer. The sample counter comparison unit 186 compares the sample counter/pointer 182 to the transmit sample preset 188, then it sends an sample increment signal 206 to the increment sample/pointer unit 183 to increment the sample counter/pointer 182 to the buffer with one unit. Concomitantly, a signal from the increment sample/pointer unit 183 forwards a toggle signal 197 to the toggle data unit 198 for toggling extra data in the extra data register unit 161. If, on the other hand, the comparison unit 186 determines that the sample counter/pointer 182 is equal to the transmit sample preset unit 188, the unit 186 sends a send “yes”-signal 190 to a forwarding send buffer data unit 192 that may forward through the transmitter medium 230 the infonnation in the data buffer (the buffer length may be one sample) to a signal receiver system 82. The medium 230 may be a wire, short distance radio wave or an infra-red signal or other suitable medium, and the receiver system 82 may contain an optional transit unit 16 consisting of a radio signal receiver or infra-red light receiver, or a computer 18, 36, a mobile phone 46, or other suitable receiving device etc. The send buffer data unit 192 also sends a sample counter reset signal 194 to a sample counter reset unit 196 that forwards a reset signal 195 to the sample counter/pointer 182. The sample counter reset unit 196 forwards a toggle signal 199 to the toggle data unit 198 for optionally toggling extra data in the optional channels digital data download register 161 which contains data, from for example a GPS unit, of such a total size that it has to be toggled on part of the total data at a time into the sample data. The toggle unit 198 sends forward a control signal 200 to a stop bit check unit 201 to check if the user has pressed a stop button on the measurement unit 12 that in turn activates a stop bit. If the stop-bit check unit 201 determines that the stop bit has been activated, the unit 201 sends a stop signal 202 to a stop unit 204 to stop the operation of the start/activation unit 102, i.e., the operation of the measurement unit 12.

If the stop-bit check unit 201 determines that the user has not activated the stop button, then the unit 201 sends a stop “no”-signal 208 to the stop button check 210 that determines if the user has pressed the stop button on the measurement unit 12. If the stop button has been activated then the stop button check 210 sends a stop button “yes”-signal 212 to a set stop bit unit 214 that through the stop flag signal 216 sets the stop bit in the unit 12 base register 140. If the stop button check 210 determines that the stop button has not been pressed then the unit 210 sends a stop “no”-signal to a reset button check 218 that determines if the reset button has been pressed. If the reset button check 218 determines that the reset button has not been pressed then the unit 218 sends a stop/reset “no”-signal 220 to the start/activation unit 102 to permit the system to continue taking samples. If the reset button check 218 determines that the reset button has been set, the unit 218 sends a reset “yes”-signal 222 to a reset process unit 224 that resets the system.

Reference figures

| | |
|---|---|
| 10 | measurement system |
| 12 | measurement unit |
| 13 | log unit key board module |
| 14 | optional log unit |
| 15 | log data module |
| 16 | optional transit unit |
| 17 | optional log data transmit module |
| 18 | computer |
| 20 | sensors |
| 21 | sensors |
| 22 | channel #1 measurement module |
| 24 | measurement module |
| 26 | process module |
| 28 | signal formatting module |
| 30 | transmitter module |
| 34 | Internet |
| 36 | computer |
| 44 | measurement module |
| 46 | mobile telephone |
| 70 | start/activation unit |
| 72 | timer unit |
| 74 | optional basic data module |
| 76 | optional buffer handling unit |
| 78 | stop function unit |
| 80 | optional reset function unit |
| 82 | receiving system |
| 100 | measurement system |
| 102 | start/activation unit |
| 104 | continous activation signal |
| 106 | clock device |
| 108 | increment signal |
| 110 | increment device |
| 111 | increment signal |
| 112 | time counter |
| 135 | “download all registers” signal |
| 113 | trace signal |
| 114 | time tracer |
| 116 | interval signal |
| 118 | interval increment device |
| 120 | interval increment signal |
| 122 | interval counter |
| 124 | interval comparison signal |
| 126 | an interval comparison unit |
| 127 | preset interval unit |
| 128 | interval equal “yes”-signal |
| 129 | continuation interval equal “no” signal |
| 130 | reset interval counter unit |
| 132 | reset interval counter signal |
| 134 | load activate signal |
| 135 | “download all registers” signal |
| 136 | “download all registers” unit |
| 137 | unit 12 ID code |
| 138 | unit 12 ID register |
| 139 | channel #1 ID code |
| 140 | unit 12 base register |
| 141 | optional channels ID code |
| 142 | optional channels ID register |
| 143 | optional channels digital data register |
| 145 | channel #1 ID register |
| 146 | channel #1 digital device |
| 147 | channel #1 digital data register |
| 148 | optional channels digital device |
| 149 | analog-to-digital converter |
| 151 | channel #1 analog device |
| 150 | log unit ID register |
| 152 | log unit ID code |
| 153 | optional channels analog device |
| 154 | log unit key register |
| 156 | unit 12 ID download register |
| 158 | unit 12 base download register |
| 160 | optional channels ID download register |
| 161 | optional channels digital data download register |
| 162 | time tracer download register |
| 164 | channel #1 ID download register |
| 166 | channel #1 digital data download register |
| 168 | log unit ID download register |
| 170 | log unit key download register |
| 172 | download validity register |
| 174 | validity signal |
| 176 | validity comparison unit |
| 178 | validity “no”-signal |
| 180 | validity “yes”-signal |
| 181 | load buffer unit |
| 182 | sample counter/pointer |
| 183 | increment sample/pointer unit |
| 186 | sample counter comparison unit |
| 188 | transmit sample preset |
| 190 | send “yes”-signal |
| 192 | send buffer data unit |
| 194 | sample counter reset signal |
| 195 | reset signal |
| 196 | sample counter reset unit |
| 197 | toggle signal |
| 198 | toggle data unit |
| 199 | toggle signal |
| 200 | control signal |
| 201 | stop bit check unit |
| 202 | stop signal |
| 204 | stop unit |
| 206 | sample increment signal |
| 208 | stop “no”-signal |
| 210 | stop button check |
| 212 | stop button “yes”-signal |
| 214 | set stop bit unit |
| 215 | process termination unit |
| 216 | stop flag signal |
| 218 | reset button check |
| 220 | stop/reset “no”-signal |
| 222 | reset “yes”-signal |
| 224 | reset process unit |
| 230 | transmitter medium |
| 240 | log unit start/activation unit |
| 242 | log key check unit |
| 244 | log stop key check |
| 250 | log key codes register |
| 252 | logical signal unit |
| 254 | wave modulator unit |
| 300 | analog timer unit |
| 302 | calibration unit |
| 304 | calibration unit |
| 306 | calibration unit |
| 310 | amplifyer |
| 312 | digital one bit converters |
| 314 | logical signal unit/s/ |
| 316 | wave modulator unit |
| 320 | amplifyer |
| 322 | digital one bit converters |
| 324 | logical signal unit/s/ |
| 326 | wave modulator unit |
| 330 | wave generator |
| 332 | wave distribution unit |
| 334 | summating unit 334 |
| 336 | single summated output signal |
| 338 | transmitter unit§ |

1. Method for interchanging, between a first subject and at least one second subject via a communication network system in real time, psychophysiological data and associated subject related data, where said first and second subjects are living beings, preferably human beings and said subjects located close to each other or scattered at different locations for achieving according to the method psychophysiological intersubject interaction, the method comprising:
collecting psychophysiological data from a first subject,
collecting first subject associated data, if any,
processing said collected first subject data in a first communication platform.
communicating said processed first subject data via said communicating network system to said second subject,
presenting said first subject psychophysiological data to said second subject as any of:
a symbolical, a functional or physical representation
collecting psychophysiological data from said second subject when reacting on said representation,
collecting said second subject associated data, if any,
processing said collected second subject data in a second communication platform,
communicating said processed second subject data via said communicating network system back to said first subject and
presenting said second subject psychophysiological data to said first subject as any of:
a symbolical, a functional or a physical representation.

2. Method according to claim 1, wherein the method includes the step:
arranging for communication of said data over a communication network system being any from the group:
Internet or other world wide web and related net functions as networks connecting to
Internet from cellular telephones,
Intranets of different types,
local nets, such as within a home or neighboring houses or vehicles or similar
micronets, such as connecting a computer or mobile phone with other devices, or, interconnecting one or more small electronic devices.

3. Method according to claim 2, comprising:
said psychophysiological data including measured values based on at least any of the manifestations from the group of: electrical brain activity, sweating, heart rate, blood pressure, blood flow, respiration, eye movements, electrical and mechanical muscle activity, acceleration of body part movements.

4. Method according to claim 3, wherein subject associated data includes any of the parameters from the group of:
log data related to the subject: such as parameters describing the subject's reactions, states or behaviour,
time,
information about the subject's geographical position,
light exposure,
ambient temperature,
climatic circumstances, such as: wind, air humidity,
surrounding sound levels,
subject affecting forces;
subject movement characteristics,
other environmental data relevant for the situation in which the subject is.

5. Method according to claim 4, wherein psychophysiological intersubject interaction includes any of the activities from the group of:
subject interacting in any of the activities from the group of: digital games, such as action games, strategic, role, sport, racing and family games, childrens' games, any other type of digital game to which psychophysiological signals may be implemented as active parts or added to in stand-alone embodiments,
chatting, i.e written or vocal or symbolical exchange of information over said network between said subjects for interpersonal communication,
group conversations, such as interactive role playing, theatre, drama or any other similar situation,
interactive happenings, such as art exhibitions,
entertainments, such as interactive television programs, musical entertainments, contests, interviews, political events,
emotional strategy games or exercises and
any other situation in which said two or more subjects reveal their psychophysiological reactions or states for each other.

6. Method according to claim 1, wherein said first or second communication platform is any from the group of:
a computer,
a game console including a special hardware design for use in digital games,
a cellular telephone,
a mobile intelligent screen or electronic book or its equivalence for local or net communication.

7. Method according to claim 1, wherein said symbolical representation is effectuated as a sign presented to the respective subject as any from the group of: a vision, a taste, a smell, a sound, a feeling.

8. Method according to claim 1, wherein said symbolical representation is effectuated as any from the group of:
a picture,
a verbal expression in text or sound,
a diagram,
a color,
an acoustic code/signal,
any other symbolic way.

9. Method according to claim 1, wherein said functional representation is effectuated as a change in the function of any from the group of:
a computer program or routine,
a cellular phone,
a communication device,
any other device or process that brings information to said first or second subject.

10. Method according to claim 1, wherein said physical representation is effectuated as a change in physical effect applied by a device attached to the respective subject's body.

11. Method according to claim 10, wherein said device is any from the group of: a vibrating device, a tapping device, a mechanical effector device, such as a device exerting mechanical pressure or movement on the subject, a heating device, a cooling device, an electrical stimulation device, any device causing some type of sensation to the subject.

12. Device for performing the method according to claim 1, characterised in that the device comprises:
a first measuring unit (12) provided with sensors (20, 21) for measuring first psychophysiological data from a first subject, a first transit unit (16, 38) receiving said first data from the first measuring unit (12) and for sending the received first data to a first communication platform (18, 36, 46) for processing the data before the data is sent over a communicating network system (34), a second communication platform (18, 36, 40, 46) for receiving said first data, a first presentation unit (18, 36) for presenting to a second subject said first data as any of a symbolical, a functional or a physical representation and
a second measuring unit (12) provided with sensors (20, 21) for measuring second psychophysiological data from said second subject, a second transit unit (16, 38) receiving said second data from the second measuring unit (12) and for sending the received second data to a third communication platform (18, 36, 46) for processing the data before the data is sent over the communicating network system (34), a fourth communication platform (18, 36, 40, 46) for receiving said second data, a second presentation unit (18, 36) for presenting said second data to said first subject as any of a symbolical, a functional or a physical representation.

13. A device according to claim 12, characterised in that the communication platform (18, 36, 46) is a computer, a cellular telephone or any other computerized communication device.

14. A device according to claim 12, characterised in that the communicating network is the world wide web, an intranet, a local net or a micronet.

15. A device according to claim 12, characterised in that any or all of the measuring unit (12), the transit unit (16, 38) and the communication platform (18, 36, 46) are housed in the same unit or cabinet at any or all of said subjects.

16. A device according to claim 12, characterised in that the first and forth communication platforms are the same.

17. A device according to claim 12, characterised in that the second and the third communication platforms are the same.

18. A device according to claim 12, characterised in that a log unit (14) is provided together with the measurement unit (12) for logging data associated to the subject being connected to the measurement unit (12).

19. A device according to claim 13, characterised in that the log unit (14) is receiving data from the measurement unit (12) and sending data to the transit unit (16, 38) by means of wired or wireless connection.

20. A device according to claim 12, characterised in that the measurement unit (12) has a plurality of sensors (20, 21) to be attached to any part of the human body of a subject for measuring or analysing psychophysiological parameters.

21. A device according to claim 20, characterised in that the measurement unit (12) has several modules, including at least any from the group of: signal amplifiers (22), amplifiers (24) designed to measure paraphysiological data, process modules (26) such as start/activation units, timer unit (72), analog to digital converter function units, basic data unit (74), final logics unit (76), stop unit (78), reset unit (80), signal formatting module (28), buffer unit, data transmitter module (30), power supply module (32).

22. A method for interchanging, between a first subject and at least one second subject via a communication network system in real time, psychophysiological data and associated subject related data, where said first and second subjects are living beings, preferably human beings and said subjects located close to each other or scattered at different locations for achieving psychophysiological intersubject interaction, characterized in that the psychophysiological data measured on the subject as analog signals are converted to digital signals by one-bit analog to digital conversion by a continuous binary summation of an amplitude-modulated electrical wave.

23. A method according to claim 22, wherein the amplitude of the wave is dependent on the order number of the channel number generating the signal for the moment.

24. A method according to claim 23, wherein the modulation of the wave is activated by logical high signals from different information sources.

25. A method according to claim 24, wherein the amplitude-modulated signals are continuously added to each other into one single signal comprising the sum of all binary amplitude-modulated single channels.
